(19) United States
(12) Patent Application Publication
(10) Pub. No.: US 2013/0204312 A1
(43) Pub. Date: Aug. 8, 2013

(54) SYSTEMS AND METHODS FOR CONTROLLING PACING INDUCED DYSSYNCHRONY TO REDUCE ISCHEMIC INJURY USING AN IMPLANTABLE MEDICAL DEVICE

(71) Applicants: Jong Gill, Valencia, CA (US); Kwangdeok Lee, Valencia, CA (US); Kyungmoo Ryu, Palmdale, CA (US); Gene A. Bornzin, Simi Valley, CA (US)

(72) Inventors: Jong Gill, Valencia, CA (US); Kwangdeok Lee, Valencia, CA (US); Kyungmoo Ryu, Palmdale, CA (US); Gene A. Bornzin, Simi Valley, CA (US)

(73) Assignee: PACESETTER, INC., Sylmar, CA (US)

(21) Appl. No.: 13/365,061

(22) Filed: Feb. 2, 2012

(51) Int. Cl. A61N 1/365

(52) U.S. Cl. 607/18; 607/17

(57) ABSTRACT

Techniques are provided for use by an implantable medical device for optimizing the amount of ventricular dyssynchrony induced within a patient during protective pacing. In one example, the device analyzes intracardiac electrogram signals to detect an ischemic event within the heart. The device then delivers pacing stimulus in accordance with adjustable pacing parameters to induce ventricular dyssynchrony within the heart and adjusts the pacing parameters within a range of permissible values to achieve a preferred degree of ventricular dyssynchrony within the patient, so long as there is no significant reduction in left ventricular pumping functionality. Preferably, the pacing parameters are adjusted to maximize or otherwise optimize the degree of dyssynchrony induced within the patient. If a significant reduction in LV pumping functionality is detected, the dyssynchrony-inducing pacing is preferably suspended to avoid any deterioration in the condition of the heart. Techniques for detecting early onset of ischemia are also disclosed.

### FIELD OF THE INVENTION

The invention generally relates to implantable cardiac stimulation devices such as pacemakers, implantable cardioverter-defibrillators (ICDs) and cardiac resynchronization therapy (CRT) devices and, in particular, to techniques for controlling pacing-induced dyssynchrony to reduce ischemic injury to the cardiac tissue including injury due to myocardial infarction.

### BACKGROUND OF THE INVENTION

Cardiac ischemia is a condition whereby heart tissue does not receive adequate amounts of oxygen and is usually caused by a blockage of an artery leading to the heart tissue. Both the ischemia itself and the reperfusion of tissues with blood following the ischemia can damage tissue. If sufficiently severe, an ischemia can result in an acute myocardial infarction (AMI), also referred to as a heart attack. With AMI, a substantial portion of heart muscle ceases to function because it no longer receives oxygen, usually due to significant blockage of the coronary artery. Generally, AMI occurs when plaque (such as fat, cholesterol, and calcium) builds up and then ruptures in the coronary artery, allowing a blood clot or thrombus to form. Eventually, the blood clot completely blocks the coronary artery and so heart tissue beyond the blockage no longer receives oxygen and the tissue dies.

Various studies have demonstrated that heart tissue can be protected from the detrimental effects of prolonged ischemia and subsequent reperfusion by subjecting the myocardium to certain prophylactic pre-conditionings prior to the ischemia. Such pre-conditioning can include the inducement of brief ischemia, the application of rapid pacing, and the inducement of an increased cardiac preload or afterload. (See, Murry et al. “Preconditioning with Ischemia: A Delay of Lethal Cell Injury in Ischemic Myocardium” Circulation 1986; 74:1124-1136; Vegh et al. “Transient Ischemia Induced by Rapid Cardiac Pacing Results in Myocardial Preconditioning” Cardiovasc Res. 1991; 25:1051-1053; and Szilvassy et al. “Ventricular Overdrive Pacing-induced Anti-ischemic effect: A Conscious Rabbit Model of Preconditioning” Am J. Physiol. 1994; 266:H2033-H2041.) However, these forms of pre-conditioning can be detrimental to cardiac hemodynamics and/or difficult to implement.

Recently, it has been demonstrated that pre-conditioning in the form of intermittent dyssynchrony induced by left ventricular (LV) pacing can have a cardiac protective effect that results in significant reduction in the infarct area of the LV wall. (See, Vanagt et al. “Pacing-Induced Dys-Synchrony Preconditions Rabbit Myocardium Against Ischemia/Reperfusion Injury” Circulation 2006; 114; I-264-I-269.) Furthermore, dyssynchrony-inducing pacing was shown to have similar protective effect when administered during the reperfusion to limit myocardial damage against the reperfusion injury. (See, Vanagt et al., “Pacing-Induced Dyssynchrony During Early Reperfusion Reduces Infarct Size” JACC 49; 17, 2007 1813-1819.)

Accordingly, pre-conditioning and post-conditioning via pacing-induced dyssynchrony is a promising technique for mitigating the effects of ischemia. Herein, techniques to induce dyssynchrony to mitigate ischemia are generally referred to as protective pacing techniques. It would be desirable to improve the efficacy of the protective pacing and aspects of the invention are directed to that end, especially for use by implantable medical devices. Moreover, many ischemic events are silent or occur suddenly making it difficult to determine the proper timing for delivery of protective pacing. It would be desirable to provide improved techniques for activating and de-activating protective pacing, and it is to that end that other aspects of the invention are directed. Still further, it is desirable to apply protective pacing using devices equipped for multi-site LV (MSLV) pacing.

### SUMMARY OF THE INVENTION

In an exemplary embodiment, a method is provided for use with an implantable cardiac stimulation device having leads for sensing and pacing the heart. The device senses cardioelectric signals within the heart such as intracardiac electrogram (IEGM) signals and analyzes the signals to detect an indication of an ischemic event within the heart. The device then delivers protective pacing stimulus in accordance with adjustable pacing parameters to induce ventricular dyssynchrony within the heart and adjusts the pacing parameters within a range of permissible values while the pacing stimulus is delivered to achieve a preferred degree of ventricular dyssynchrony within the patient, so long as there is no significant drop in LV pumping functionality. For example, the pacing parameters can be adjusted to maximize or otherwise optimize the degree of dyssynchrony induced within the patient. Thus, rather than merely inducing some general and nonspecific increase in ventricular dyssynchrony, the present technique (in at least some embodiments) serves to adjust pacing control parameters to maximize the ventricular dyssynchrony. If a significant reduction in LV pumping functionality is detected, the dyssynchrony-maximizing pacing is preferably suspended to avoid any deterioration in the condition of the heart.

In accordance with various illustrative embodiments, the dyssynchrony-maximizing pacing may be delivered prior to the onset of full ischemia (pre-conditioning pacing), during the episode of full ischemia (therapeutic ischemic pacing) and/or during a reperfusion phase following the episode of full ischemia (post-conditioning pacing.) Within each of these phases it may be advantageous to selectively adjust one or more of: the pacing configuration (e.g. biventricular vs. MSLV pacing); the pacing location (particularly within the LV); the pacing rate; the atrioventricular (AV/PV) pacing delay interval; the interventricular (VV) pacing delay interval; interpulse intervals used with MSLV; and/or the duration of sequences of the protective pacing stimulus. Different protective pacing strategies or “protocols” may be exploited within each of these phases using different combinations of pacing parameters to achieve pre-programmed goals. For example, the pre-conditioning protocol can differ from the reperfusion protocol. Moreover, the number of electrodes used for pacing can be selected from multiple LV electrodes.

To time the delivery of pre-conditioning stimulus, the device preferably detects early onset of an ischemia by comparing deviation in ST segment elevation against a relatively low ST deviation threshold (Threshold_{PRE}) indicative of early onset of ischemia (where the ST segment is defined, e.g., as the portion of the IEGM signal between the end of a QRS complex and the beginning of a T-wave.) Threshold_{PRE} is set lower than the threshold used to subsequently detect full onset of an episode of ischemia (Threshold_{FULL}). If the ST change begins to reduce during the course of delivery of pre-conditioning stimulus, it is possible that the delivery of preconditioning stimulus is terminated. In this manner, the device effectively “predicts” the full onset of ischemia before it occurs, thereby allowing pre-conditioning pacing to be delivered in a timely manner. Pre-conditioning may be applied, for example, in alternating cycles of five minutes of dyssynchrony-maximizing protective pacing followed by five minutes of conventional pacing or no pacing. It is also feasible that the duration of preconditioning pacing is limited to programmable duration to prevent potential detrimental effect to the function of heart. To time the delivery of post-conditioning stimulus, the device may use a reperfusion threshold (Threshold_{REPERFUSION}) indicative of the reperfusion phase following ischemia. The detection of the reperfusion phase can be made based on the trajectory of ST deviation trending, e.g., the slope of ST deviation is negative. Threshold_{REPERFUSION} is preferably set well below Threshold_{FULL}. Once the ST deviation drops below Threshold_{REPERFUSION}, the reperfusion pacing protocol is activated. Reperfusion pacing may be applied, for example, for a period of two minutes following detection of the reperfusion phase.

In one example, to maximize ventricular dyssynchrony, the device continuously or periodically assesses electrical and/or mechanical dyssynchrony based on IEGM signals, cardiogenic impedance signals or other suitable parameters such as changes in activation interval (AI) or increases in preload as detected based on heart sounds or photoplethysmography (PPG) signals obtained using implanted sensors. While the amount of dyssynchrony is assessed, the device automatically adjusts pacing parameters (such as pacing delays or pacing locations) in a closed feedback loop to identify pacing parameters that tend to maximize ventricular dyssynchrony (at least within acceptable ranges of those parameters.) Concurrently, the device assesses LV pumping functionality based, e.g., on contractility proxies to detect any significant drop in pumping functionality. If such a drop is detected, the pacing to induce ventricular dyssynchrony is suspended or adjusted to be less aggressive.

System and method implementations of these and other techniques are presented herein.

### BRIEF DESCRIPTION OF THE DRAWINGS

Features and advantages of the described implementations can be more readily understood by reference to the following description taken in conjunction with the accompanying drawings.

FIG. 1 illustrates components of an implantable medical system having a pacemaker, ICD or CRT device equipped with a therapeutic ventricular dyssynchrony-maximizing system for use in selectively inducing dyssynchrony prior to, during, and after an episode of cardiac ischemia;

FIG. 2 summarizes a general technique for inducing dyssynchrony to reduce ischemic injury that may be performed by the device of FIG. 1, where pacing parameters are adjusted to achieve a preferred or optimal degree of dyssynchrony;

FIG. 3 illustrates an exemplary technique for use with the general method of FIG. 2, wherein a set of ST deviation thresholds of differing magnitude are employed to time and control the delivery of pacing-induced dyssynchrony;

FIG. 4 graphically illustrates the set of ST deviation thresholds employed by the technique of FIG. 3 for use in detecting an episode ischemia;

FIG. 5 illustrates a pre-conditioning technique for use prior to full onset of ischemia in accordance with the technique of FIG. 3;

FIG. 6 illustrates a therapeutic pacing technique for use during an episode of full ischemia in accordance with the technique of FIG. 3;

FIG. 7 illustrates a post-conditioning technique for use during reperfusion following ischemia in accordance with the technique of FIG. 3;

FIG. 8 is an alternative representation of the technique of FIG. 3, highlighting certain feedback loops;

FIG. 9 is a simplified, partly cutaway view, illustrating the device of FIG. 1 along with at set of leads implanted in or on the heart of the patient; and

FIG. 10 is a functional block diagram of the pacer/CRT of FIG. 9, illustrating basic circuit elements that provide cardioversion, defibrillation and/or pacing stimulation in the heart and particularly illustrating components for performing the techniques of FIGS. 2-8.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Implantable System

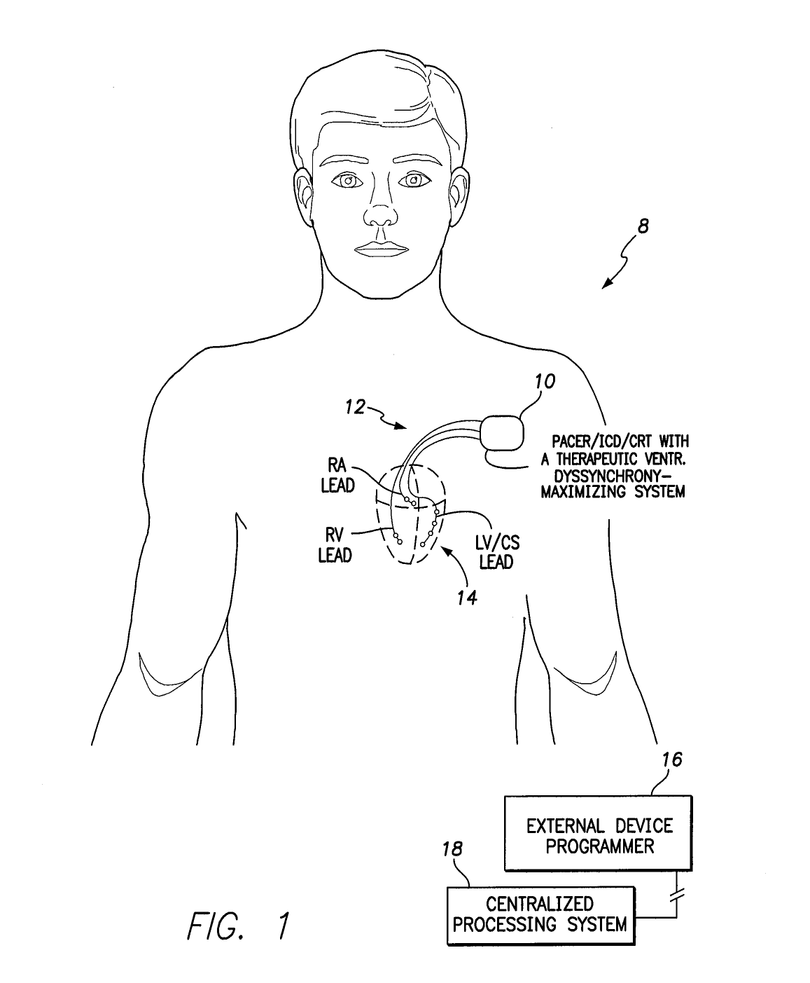

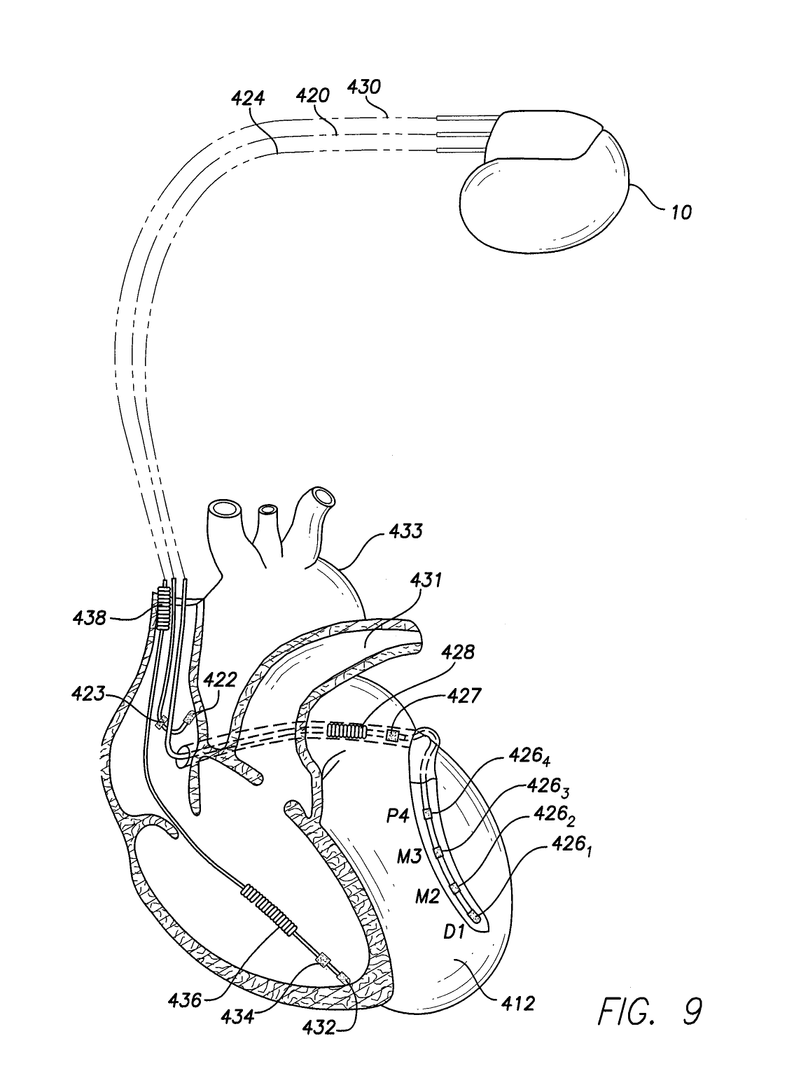

FIG. 1 illustrates an implantable medical system 8 equipped with a therapeutic ventricular dyssynchrony-maximizing system for use in selectively inducing dyssynchrony prior to, during, and after an episode of cardiac ischemia. In this particular example, the implantable medical system 8 includes a pacer/ICD/CRT 10 or other implantable cardiac rhythm management device equipped with a set of cardiac sensing/pacing leads 12 implanted on or within the heart of the patient, including a multi-pole LV lead implanted via the coronary sinus (CS). To illustrate the multi-pole configuration of the LV lead, a set of electrodes 14 is shown distributed along the LV lead. In FIG. 1, a stylized representation of the set of leads is provided. A more accurate illustration of the LV lead is provided within FIG. 9, discussed below, which includes a distal (D1) electrode, a first ring electrode (M2), a second ring electrode (M3) and a proximal ring electrode (P4.)

In the examples described herein, a quad-pole (or “quadrapolar” or “quadripolar”) lead is employed, such as the Quartet™ lead provided by St Jude Medical. Other suitable leads may instead be employed, including leads with more or fewer electrodes. Also, as shown, an exemplary RV lead is provided that includes an RV tip/ring electrode pair. An RA lead is also provided that includes an RA tip/ring pair. Other electrodes of various sizes and shapes may be additionally or alternatively provided, such as various coil electrodes for delivering shock therapy. Although identified as a “pacer/ICD/CRT” in FIG. 1, it should be understood that device 10 can be any suitably-equipped implantable medical device, such as a standalone pacemaker, ICD or CRT device, including CRT-D and CRT-P devices. In the following, for brevity, device 10 will be referred to simply as a pacer/CRT.

The pacer/CRT may be used in conjunction with an external device programmer 16, which allows a clinician to program the device. Note also that other external systems might instead be used such as bedside monitors or the like. In some embodiments, the external system is directly networked with a centralized computing system 18, such as the HouseCall™ system or the Merlin@home—Merlin.Net systems of St. Jude Medical.

### Summary of Protective Pacing Techniques for Inducing Dyssynchrony

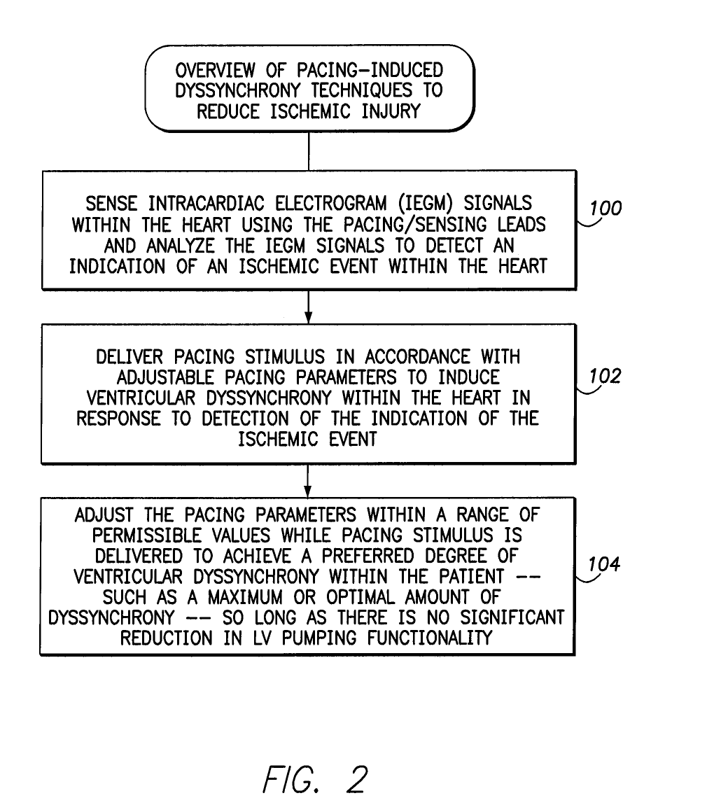

FIG. 2 broadly summarizes techniques exploited by the pacer/CRT of FIG. 1 (or other suitably-equipped systems) for maximizing or optimizing ventricular dyssynchrony as a prophylactic measure prior to an ischemia or as a therapy during or after the ischemia. Herein, the term ischemia is deemed to broadly include infarction and acute coronary syndrome (ACS). At step 100, the pacer/CRT senses IEGM signals within the heart using the pacing/sensing leads and analyzes the IEGM signals to detect an indication of an ischemic event within the heart. The indication may be, e.g., an indication of (1) early onset of a possible episode of ischemia, (2) full onset of an ischemia, and/or (3) a reperfusion phase following the end of ischemia. As will be explained below, these indications of ischemic events may be detected based on certain changes in the elevation of the ST segment.

At step 102, the pacer/CRT delivers pacing stimulus in accordance with adjustable pacing parameters to induce ventricular dyssynchrony within the heart in response to detection of the indication of the ischemic event. The pacing parameters to be adjusted include, for example, the pacing configuration (e.g. biventricular vs. MSLV pacing); the pacing location (particularly within the LV); the pacing rate; the AV/PV pacing delay interval; the VV pacing delay interval; and the duration of sequences of the prophylactic pacing stimulus, or other parameters such as interpulse intervals used with MSLV. At step 104, the pacer/CRT adjusts the pacing parameters (within respective ranges of permissible values) while pacing stimulus is delivered to achieve a preferred degree of dyssynchrony within the patient—such as a maximum or optimal amount of dyssynchrony—so long as there is no significant reduction in LV pumping functionality. As will be explained below, either electrical dyssynchrony, mechanical dyssynchrony, or both may be assessed while pacing parameters are concurrently adjusted to determine a combination of parameters that maximizes or optimizes dyssynchrony so as to provide optimal prophylactic therapy to reduce ischemic injury. In many cases, by maximizing dyssynchrony, the device thereby induces maximum stretch in the late activated infarct area to reduce ischemic injury. LV pumping functionality may be concurrently assessed based on contractility measurements or proxies or using other suitable parameters. If LV pumping functionality falls below an acceptable level, the protective pacing may be suspended or its aggressiveness reduced.

It should be understood that the “optimal” degree of dyssynchrony obtained using techniques described herein is not necessarily absolutely optimal in a given quantifiable or mathematical sense. What constitutes “optimal” depends on the criteria used for judging the resulting performance, which can be subjective in the minds of some clinicians. The degree of dyssynchrony achieved by the techniques described herein represents, at least, a “preferred” degree of dyssynchrony. Clinicians may choose to adjust or alter the pacing parameters used to achieve the preferred degree of dyssynchrony for particular patients at their discretion.

### Exemplary Techniques for Controlling Protective Pacing

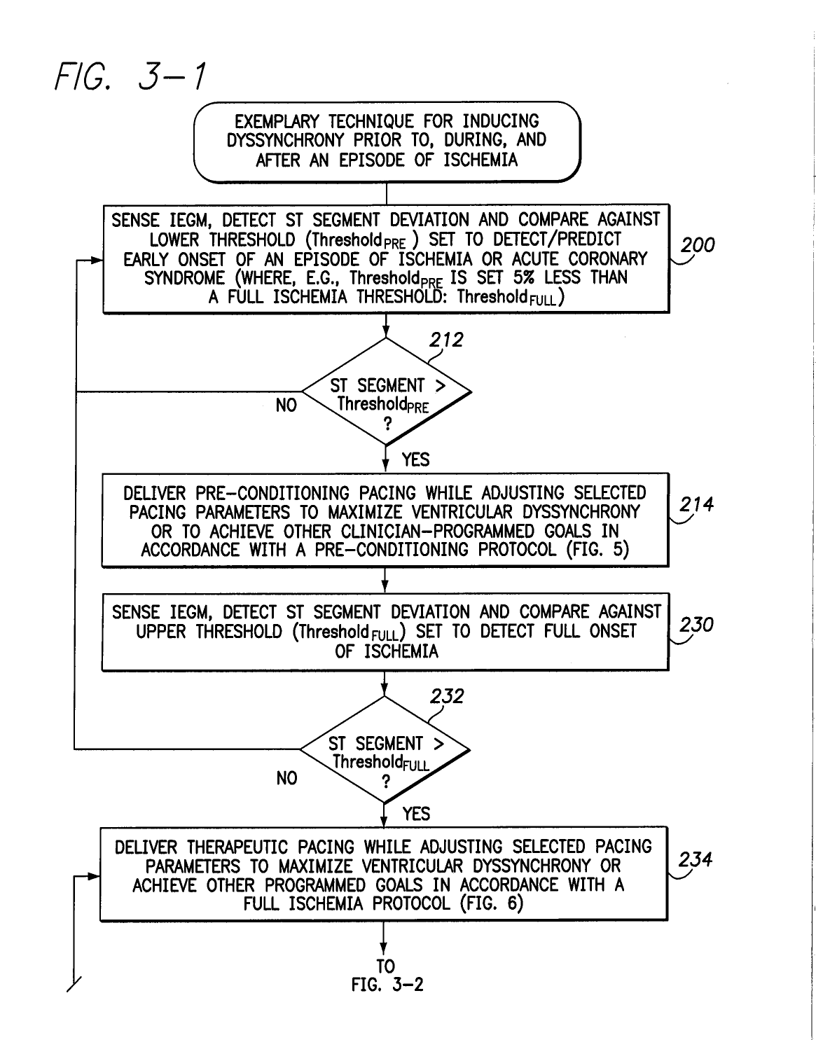

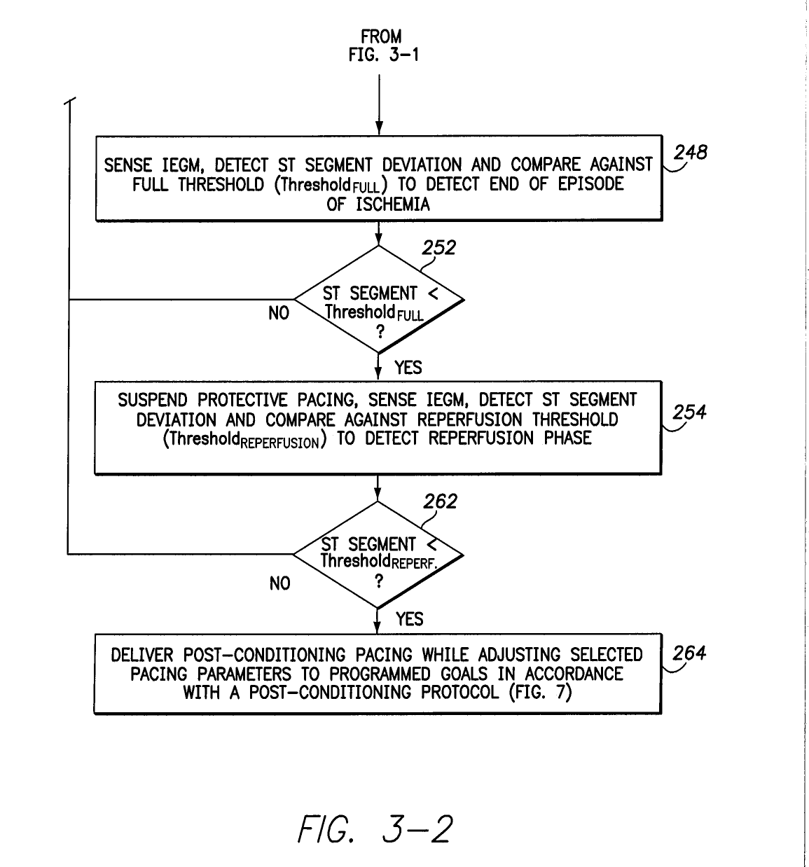

FIGS. 3-8 illustrate exemplary techniques for activating and controlling Pacing-Induced Dyssynchrony. Beginning at step 200 of FIG. 3, the pacer/CRT senses one or more IEGM signals, detects ST segment deviation and compares the deviation against a lower threshold (Threshold_{PRE}) set to detect/predict early onset of an episode of ischemia, infarction or ACS. In one example, Threshold_{PRE} is set 5% less than the threshold used to detect the onset of a full episode of ischemia (Threshold_{FULL}.) The amount of ST deviation can be calculated and tracked using otherwise conventional techniques. See, for example, techniques set forth in: U.S. Pat. No. 7,297,114 to Gill et al., entitled “System and Method for Distinguishing among Cardiac Ischemia, Hypoglycemia and Hyperglycemia using an Implantable Medical Device”; U.S. Pat. No. 7,792,572 to Gill et al., entitled “Ischemia Detection using Intra-Cardiac Signals”; U.S. Pat. No. 7,769,436 to Boileau et al., entitled “System and Method for Adaptively Adjusting Cardiac Ischemia Detection Thresholds and Other Detection Thresholds used by an Implantable Medical”; U.S. Pat. No. 7,610,086 to Ke et al., entitled “System and Method for Detecting Cardiac Ischemia in Real-Time using a Pattern Classifier Implemented within an Implanted Medical Device;” U.S. Pat. No. 7,865,232 to Krishnaswamy et al., entitled “Method and System for Automatically Calibrating Ischemia Detection Parameters”; and U.S. Pat. No. 7,725,171 to Zhu et al., entitled “System and Method for Tracking ST Shift Data utilizing Histograms.” Preferably, though, the ST segment is tracked using the AnalyST™ system of St. Jude Medical.

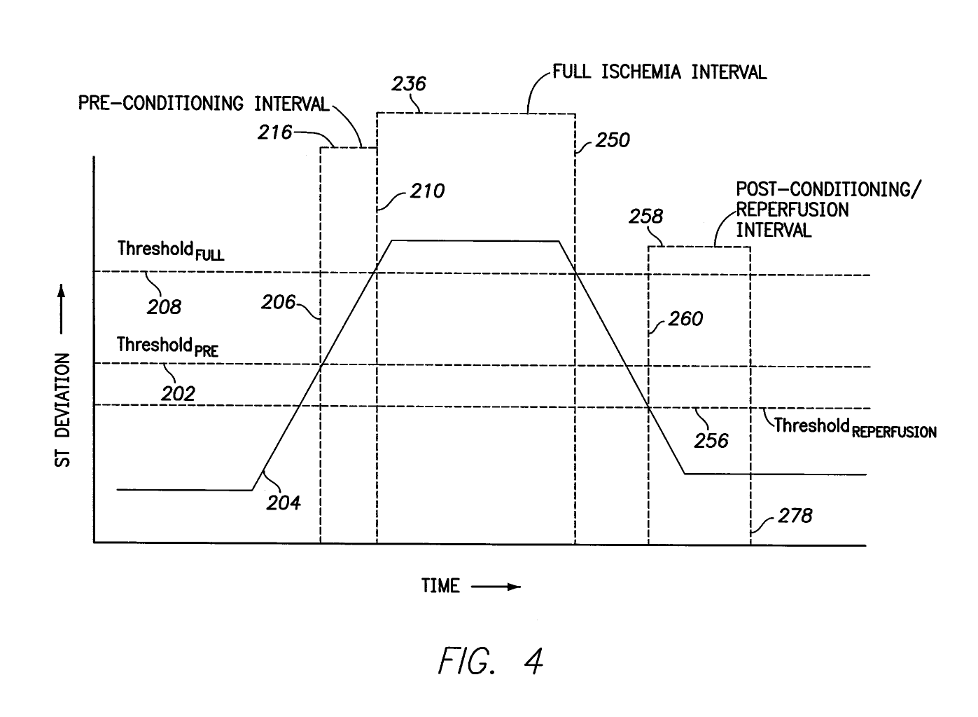

FIG. 4 illustrates Threshold_{PRE} by way of line 202. Early onset of ischemia is indicated when the ST deviation 204 exceeds the threshold at time 206. This may also be regarded as predicting the subsequent ischemia, which is not deemed to fully occur until the ST deviation exceeds the higher Threshold_{FULL} 208 and time 210. (Note that the difference between Threshold_{PRE} and Threshold_{FULL} is exaggerated within the drawing for clarity. As noted, the actual difference might be only 5%.) Returning to FIG. 3, if the ST deviation detected at step 200 exceeds Threshold_{PRE} at step 212, then pre-conditioning processing begins at step 214 where the device delivers pre-conditioning pacing while adjusting selected pacing parameters to maximize ventricular dyssynchrony or to achieve other clinician-programmed goals in accordance with a pre-conditioning protocol. The pre-conditioning phase or interval is shown in FIG. 4 by way of interval 216.

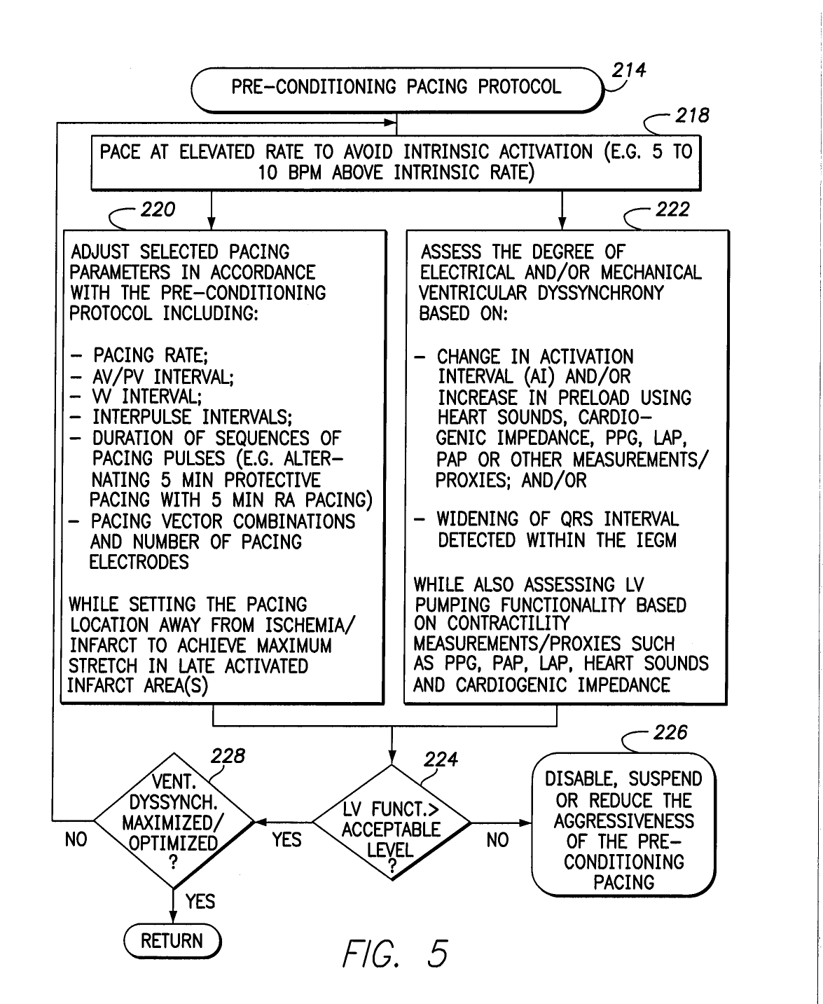

FIG. 5 illustrates an exemplary pre-conditioning protocol 214. During pre-conditioning, the device paces the ventricles at step 218 at an elevated rate to avoid intrinsic activation, such as at a rate 5 to 10 beats per minute (bpm) above intrinsic rate. Then, at step 220, the device begins to adjust selected pacing parameters in accordance with the pre-conditioning protocol including one or more of: the pacing rate; the AV/PV interval; the VV interval; interpulse MSLV intervals; the duration or periodicity of sequences of pacing pulses (e.g. the device might be programmed to alternate five minutes of protective pacing with five minutes of conventional RA pacing;); all possible pacing vector combinations; and the number of pacing electrodes.

The particular parameters to be adjusted (and the order in which they are adjusted) can be selected in advance by the clinician and programmed into the device. For each parameter, the device or the clinician specifies a range of permissible values, such as a range of permissible pacing rates, AV/PV delay values, etc. The device adjusts the parameters within their respective permissible ranges while also setting or adjusting the pacing location (by, e.g., selecting a particular LV location for delivery of stimulation via a quad-pole LV lead) to a site away from the expected location of the ischemia/infarct so as to achieve maximum stretch in late activated infarct area(s) without significantly increasing the wall stress in the infarct area. For example, if the ischemia is expected to occur near the apex of the ventricles, the device might set the device to deliver pacing via the proximal (P4) electrical of the quad-pole LV lead. Conversely, if the ischemia is expected to occur near the AV groove, the device might set the device to deliver pacing via the distal (D1) electrical of the quad-pole LV lead. In this regard, site-specific pacing may be particularly beneficial when the infarct border zone rather than remote area is paced at this early stage of AMI, which would attenuate remodeling. For example, if the ischemia is expected to occur near the apex of the LV, the device delivers the preventive pacing that could unload areas of high wall stress and induce adequate dyssynchrony at the same time.

Insofar as setting the pacing location is concerned, the expected location of the ischemia may be determined based on known locations of past ischemic events within the patient or based on an on-going analysis of the IEGM signals. See, for example, U.S. Pat. No. 7,908,004 to Gill et al., entitled “Considering Cardiac Ischemia in Electrode Selection” and U.S. Pat. No. 7,856,268 to Kroll et al., entitled “Ischemia Detection for Anti-Arrhythmia Therapy.” If the expected location of the ischemia is not known or cannot be determined, the device may set the pacing location to a default location, such as the location of the D1 electrode.

Concurrently, at step 222, the device assess the degree of electrical and/or mechanical ventricular dyssynchrony based on: a change in AI and/or an increase in preload by using heart sounds, cardiogenic impedance, PPG signals or other suitable measurements/proxies. Dyssynchrony may also be assessed based on IEGM morphology, such as by measuring any significant widening of the QRS interval. Insofar as impedance is concerned, it should be understood that related electrical parameters might be detected and/or exploited instead, such as admittance, conductance or immittance. Those skilled in the art can convert among these related parameters as needed. Herein, “impedance” is intended to generally include related electrical parameters such as admittance, conductance and immittance. Additionally at step 222, the device assesses LV pumping functionality based on contractility measurements/proxies or other suitable proxies, including proxies based on PPG, left atrial pressure (LAP), pulmonary artery pressure (PAP), heart sounds or cardiogenic impedance. A CardioMEMS™ or similar device may be used to assess some of these parameters. See, for example, U.S. Pat. No. 7,621,036 of Cros et al., entitled “Method of Manufacturing Implantable Wireless Sensor for In Vivo Pressure Measurement,” U.S. Published Patent Application 2006/0287602 of O'Brien et al., entitled “Implantable Wireless Sensor for In Vivo Pressure Measurement,” and U.S. Pat. No. 8,021,307 to White et al., entitled “Apparatus and Method for Sensor Deployment and Fixation,” each initially assigned to CardioMems, Inc.

Various techniques for assessing dyssynchrony are discussed in: U.S. Pat. No. 7,676,264 Pillai et al., entitled “Systems and Methods for use by an Implantable Medical Device for Evaluating Ventricular Dyssynchrony based on T-wave Morphology;” U.S. Patent Application 2011/0118803 of Hou et al., entitled “Cardiac Resynchronization Therapy Optimization using Vector Measurements obtained from Realtime Electrode Position Tracking”; U.S. Patent Application 2010/0121397 of Cholette, entitled “System and Method for Evaluating Mechanical Cardiac Dyssynchrony Based on Multiple Impedance Vectors Using an Implantable Medical Device”; and U.S. Patent Application 2009/0318995 of Keel et al., entitled “Cardiac Resynchronization Therapy Optimization using Mechanical Dyssynchrony and Shortening Parameters From Realtime Electrode Motion Tracking.” See, also, U.S. patent application Ser. No. 13/217,554, filed Aug. 25, 2011 of Bornzin et al., entitled “Systems and Methods for Assessing Heart Failure and Controlling Cardiac Resynchronization Therapy using Hybrid Impedance Measurement Configurations.” (Atty. Docket No. a11p1026.)

As noted, LV pumping function may be assessed based on contractility. Techniques for detecting cardiac contractility are discussed in, e.g., U.S. Pat. No. 6,788,970 to Park, et al., U.S. Pat. No. 6,208,900 to Ecker et al. and U.S. Pat. No. 4,485,813 to Anderson et al. Heart sound waves can also be used to determine contractility and other related parameters (e.g., stroke volume, blood pressure and dP/dt), as disclosed in U.S. Pat. No. 6,044,299 to Nilsson. IEGM signals may also provide a basis for determining contractility. See, for example, U.S. Pat. No. 4,759,366 to Callaghan. Impedance measurements of blood in the heart can also been employed to derive contractility of the myocardium. See, U.S. Pat. No. 4,884,576 to Alt and U.S. Pat. No. 4,535,774 to Olsen. Also, the rate of change in impedance (dZ/dt) has been shown to correspond to contractility. See, for example, U.S. Pat. No. 4,733,667 to Olive et al. and U.S. Pat. No. 5,800,467 to Park et al. In some examples, surrogates for myocardial contractility are derived from cardiac pressure signals or PPG signals. See, for example, techniques described in published U.S. Patent Application No. 2010/0234906 of Koh, entitled “System and Method for Controlling Rate-Adaptive Pacing based on a Cardiac Force-Frequency Relation detected by an Implantable Medical Device.”

AI and dyssynchrony are discussed in U.S. Patent Application 2010/0087889 of Maskara et al., entitled “Dynamic Cardiac Resynchronization Therapy by Tracking Intrinsic Conduction,” Insofar as preload is concerned, it is know that changes in contractility can produce significant changes in ejection fraction (EF). Increasing contractility leads to an increase in EF, while decreasing contractility tends to decrease EF. Therefore, EF can be used as a clinical index for evaluating the inotropic state of the heart. In heart failure, for example, an associated decrease in contractility leads to a fall in stroke volume as well as an increase in preload, thereby decreasing EF. See, e.g., U.S. Pat. No. 7,123,961 to Kroll et al. and U.S. Pat. No. 5,549,650 to Bomzin et al.

Following steps 220 and 222, the device determines at step 224 whether LV pumping functionality remains above an acceptable level despite the increase in dyssynchrony induced by the pre-conditioning pacing. If the LV functionality has dropped below the acceptable level (as may be assessed, e.g., by comparing contractility proxies against a suitable threshold value set by the clinician), the device disables, suspends or reduces the aggressiveness of the pre-conditioning pacing at step 226 based on clinician-specified programming. Assuming that LV pumping functionality is still sufficient, the device instead assesses whether ventricular dyssynchrony has been maximized or, at least, optimized, at step 228. If not, the procedure of FIG. 5 repeats in a closed loop to make further adjustments to the pacing parameters until dyssynchrony is maximized/optimized.

Insofar as “maximizing” dyssynchrony is concerned, the device may adjust the selected pacing parameters within their respective permissible ranges until identifying a combination of parameters that yields the largest degree of ventricular dyssynchrony that can be achieved given the practical limitations of the device. In many cases, for example, pacing parameters can only be set to certain incremental values, such as certain time delay increments or pacing rate increments. That is, the pacing parameters typically cannot be set to any value within a continuous range of values but can only be set to certain discrete values, and so the maximization of dyssynchrony achieved via this procedure may be limited to what can be practically achieved given the discrete programmable values. Insofar as “optimizing” dyssynchrony is concerned, the device may adjust the selected pacing parameters within their respective permissible ranges until identifying a combination of parameters that yields an optimal degree of ventricular dyssynchrony (that can be achieved given the practical limitations of the device) as determined by optimization criteria specified in advance by the clinician and programmed into the device. Hence, in at least some examples, the device will not seek to maximize dyssynchrony but instead to achieve other clinician-specified goals. As with maximization, optimization may be limited by the practical constraints of the device. Once ventricular dyssynchrony is maximized or optimized, processing returns to FIG. 3 where the device continues to monitor ST segments to detect full onset of ischemia. In some examples, the device operates to increase the degree of dyssynchrony if the level of ischemia increases as indicated by the level of ST changes.

Returning to FIG. 3, at step 230, the device senses IEGMs, detect ST segments and compares against the upper threshold (Threshold_{FULL}) set to detect full onset of ischemia. If the ST deviation exceeds Threshold_{FULL} at step 232, then full ischemia processing begins at step 234 where the device delivers therapeutic pacing while adjusting selected pacing parameters to maximize ventricular dyssynchrony or achieve other programmed goals in accordance with a full ischemia protocol. The full ischemia phase or interval is shown in FIG. 4 by way of interval 236.

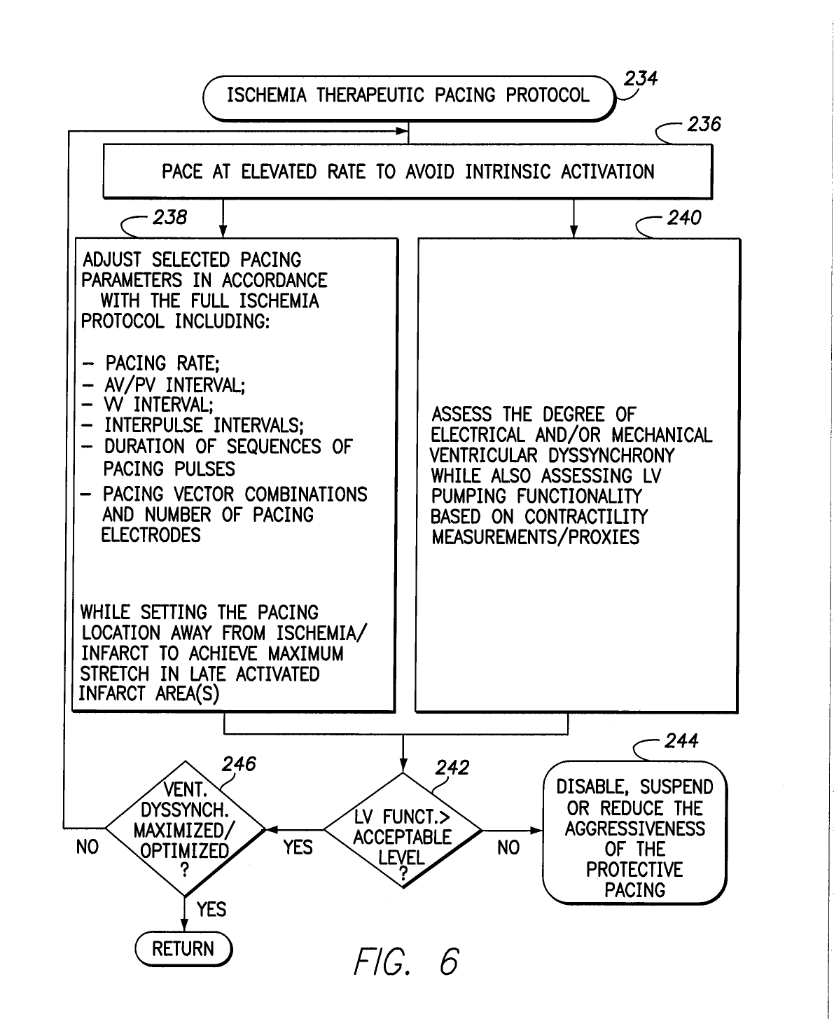

FIG. 6 illustrates a full ischemia protocol 234. Many of the steps are similar to those of the pre-conditioning technique of FIG. 5 and hence will not be described again in detail. Although the overall closed-loop procedure is similar, it is noted that the particular parameters selected by the clinician for use in maximizing or optimizing dyssynchrony during full ischemia (as well as the criteria selected for optimization) might differ from those used during pre-conditioning. As before, the device paces the ventricles (step 236) at an elevated rate to avoid intrinsic activation. At step 238, the device begins to adjust selected pacing parameters in accordance with the full ischemia protocol, which might differ from the pre-conditioning protocol. Concurrently, at step 240, the device assesses the degree of ventricular dyssynchrony and LV pumping functionality. Following steps 238 and 240, the device determines at step 242 whether LV pumping functionality remains above an acceptable level and, if not, the device disables, suspends or reduces the aggressiveness of the protective pacing delivered during full ischemia at step 244. Assuming that LV pumping functionality is still sufficient, the device assesses whether ventricular dyssynchrony has been maximized or, at least, optimized, at step 246 and, if needed, the device repeats the procedure of FIG. 6 in a closed-loop to make further adjustments to the pacing parameters until dyssynchrony is maximized/optimized. Once ventricular dyssynchrony is maximized or optimized, processing returns to FIG. 3 where the device continues to monitor ST segments to detect the end of full ischemia. Note that, in at least some examples, the protective pacing delivered during full ischemia may not be delivered throughout the episode of full ischemia but is instead delivered only during a programmable window. Also, if the same protective pacing protocol is used during pre-conditioning as during full ischemia, this may be regarded as extending pre-conditioning pacing into the full ischemia interval to overlap with at least a portion of the full ischemia interval.

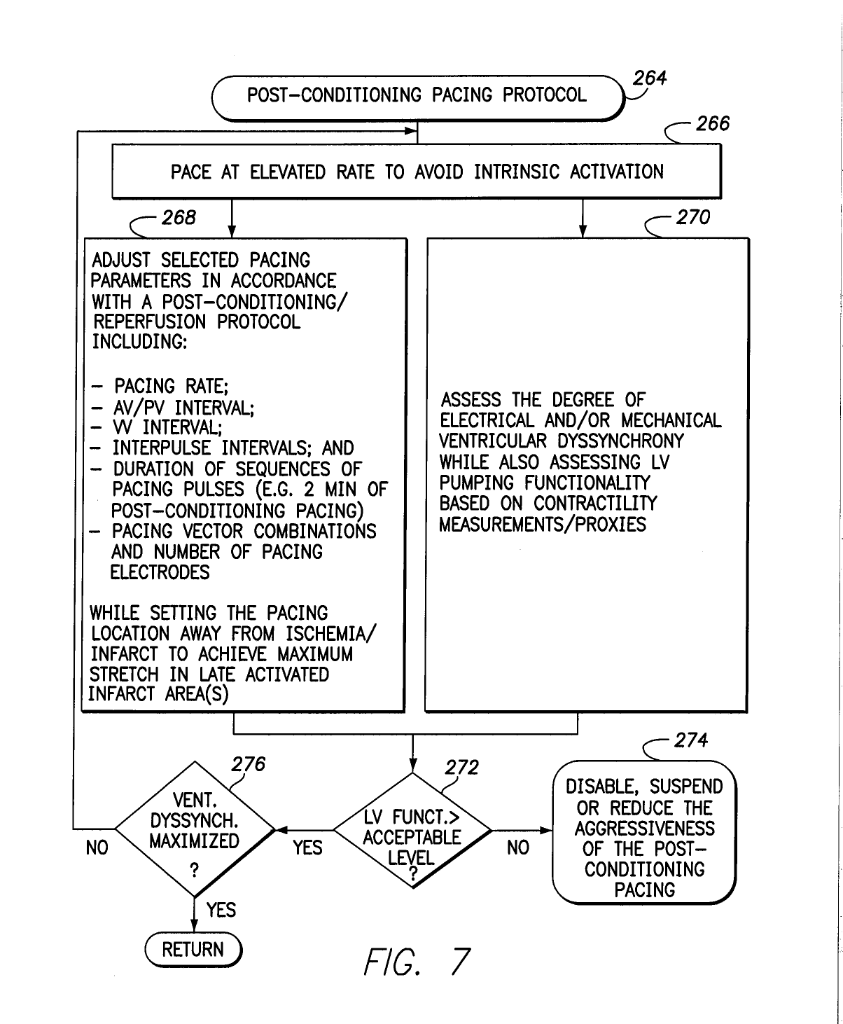

Returning to FIG. 3, at step 248, the device senses IEGMs, detects ST segment deviation and compares against Threshold_{FULL} to detect the end of the episode of full ischemia. In FIG. 4, the end of full ischemia is denoted by time 250. If at step 252, the device detects the end of the episode of full ischemia, then at step 254 the device suspends protective pacing pending the reperfusion phase when post-conditioning pacing will be delivered. That is, at step 254, the device senses IEGMs, detects ST segment deviations and compares against Threshold_{REPERFUSION}. In FIG. 4, Threshold_{REPERFUSION} is denoted 256 and the beginning of the post-conditioning interval 258 is denoted by time 260. If at step 262 of FIG. 3, the device detects that the ST deviation has fallen below the reperfusion threshold, then at step 264 the device delivers post-conditioning pacing while adjusting selected pacing parameters to programmed goals in accordance with a post-conditioning protocol (FIG. 7.)

FIG. 7 illustrates an exemplary post-conditioning protocol 264. Many of the steps are similar to those of the closed loop feedback techniques of FIGS. 5 and 6 and hence will not be described again in detail. Although the overall closed-loop procedure is similar, it is again noted that the particular parameters to be selected by the clinician for use in maximizing or optimizing dyssynchrony might differ, as well as the criteria selected for optimization. As before, the device paces the ventricles (step 266) at an elevated rate to avoid intrinsic activation. At step 268, the device adjusts selected pacing parameters in accordance with the reperfusion protocol, which might differ from both the pre-conditioning protocol and the full ischemia protocol. Concurrently, at step 270, the device assesses the degree of ventricular dyssynchrony and LV pumping functionality. Thereafter, the device determines at step 272 whether LV pumping functionality remains above an acceptable level and, if not, the device disables, suspends or reduces the aggressiveness of the post-conditioning pacing at step 274. Assuming that LV pumping functionality is still sufficient, the device assesses whether ventricular dyssynchrony has been maximized or, at least, optimized, at step 276 and, if needed, repeats the procedure of FIG. 7 in a closed-loop to make further adjustments to the pacing parameters until dyssynchrony is maximized/optimized. Post-conditioning pacing may be programmed to end at some set time, such as after two minutes of post-conditioning. Alternatively, it may be set to end sometime after ST segment deviation has stabilized. Within FIG. 4, the post-conditioning interval is shown to end a set time after it commences, as indicated by time 278.

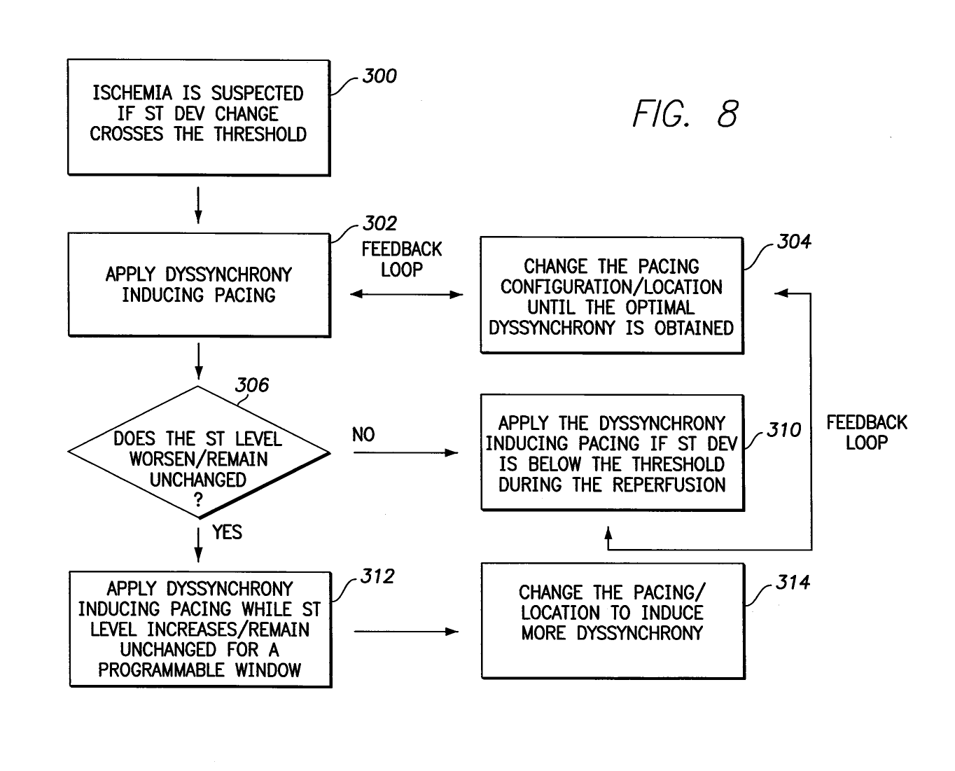

FIG. 8 illustrates exemplary protective pacing procedures performed by the pacer/CRT by way of an alternative “conceptual” flowchart that emphasizes the various feedback loops. As these procedures are similar to those that have already been described, FIG. 8 will only briefly be summarized. At step 300, pacer/CRT determines whether ischemia is suspected (i.e. it detects the onset of ischemia) if the ST deviation crosses an ischemia threshold (such as Threshold_{FULL}.) At step 302, the device applies dyssynchrony-induced pacing while, at step 304, the device changes the pacing configuration/location until optimal dyssynchrony is obtained. At 306, the device assesses whether the ST level “worsens/remains unchanged” or “improves.” If the ST levels improve (i.e. the decision of block 306 is “no” because ST deviation is decreasing), the device at step 310 applies further dyssynchrony-inducing pacing once the ST deviation is below the reperfusion threshold while also changing the pacing configuration/location (step 304) to maintain optimal dyssynchrony. Conversely, if the ST levels are worsening or remain unchanged (i.e. the decision of block 306 is “yes” because ST levels are not dropping), the device at step 312 applies further dyssynchrony-inducing pacing during a programmable window while changing the pacing configuration/location to induce even more dyssynchrony. Following step 314, processing may transition of step 310 for post-conditioning during the reperfusion phase (as already described.)

Thus, what have been described are various techniques for activating and controlling dyssynchrony-inducing protective pacing. In the following, some of the features and advantages of these techniques are summarized and elaborated.

In various exemplary embodiments, protective pacing can be administered from ventricular leads from either RV (in pacemakers and ICDs) or LV (in CRT devices.) The RV lead can also be used in ICDs and pacemakers to induce dyssynchrony. The pacing rate should be set in such a way that no intrinsic activation is present during the pacing. An exemplary pacing rate is 5-10 bpm above the intrinsic heart rate. The dyssynchrony-inducing pacing may be administered with AV or PV interval being zero. However, the AV or PV interval can be lengthened to a more physiological level as long as dyssynchronous activation is induced. As described, the dyssynchrony-inducing pacing can be delivered through a closed-loop system. For instance, the AV interval, the rate of pacing, and/or duration of pacing can be modulated until the largest degree of dyssynchrony (either mechanical or electrical) is obtained in order to achieve the highest degree of cardioprotection. Exemplary measurements for dyssynchrony include: delay in AI; an increase in preload; or a widening of QRS interval. AI and preload can be measured by heart sound, cardiogenic impedance and PPG. QRS width can be measured by multitude of unipolar EGMs. The timing of the delivery of protective pacing can be determined by the detection of ischemic event by the AnalyST™ system or other suitable system. The protective pacing may be controlled as follows:

### Pre-Conditioning:

The thresholds (ST Thresholds) of the AnalyST™ system are preferably determined so as to detect the ischemic event or ACS. In order to ensure the efficacy of the protection against myocardial injury in pre-conditioning, it is desirable that protective pacing be delivered before the full-blown ischemic event. Therefore, thresholds (Threshold_{PRE}) that are lower than ischemia detecting thresholds can be used for the delivery of pre-conditioning pacing, as discussed above. Exemplary Threshold_{PRE} is 5% below the ST thresholds used for detecting ischemia and ACS events. Furthermore, the Threshold_{PRE} values can vary in order to ensure adequate pre-conditioning effect. Intermittent protective pacing for five minutes, interspersed with five minutes of RA pacing, may be delivered until the regular ST thresholds are crossed. As already explained, the pre-conditioning pacing can extend well into the full ischemia. The pacing rate and location can be changed to induce more dyssynchrony if the effect of ischemia continues to deteriorate the condition of the heart. For instance, if LV pumping functionality is compromised (as detected by contractility sensors such as heart sound, cardiogenic impedance), the pacing configuration and location can be changed to induce more dyssynchrony to induce more cardio-protective effect. However, if the LV conditions deteriorate below certain point, the dyssynchrony-inducing pacing should terminate as it might lead to further deterioration of the condition of the heart.

### Post-Conditioning:

The same thresholds used for the pre-conditioning pacing can be used for the post-conditioning or thresholds that are lower than the Threshold_{PRE} may be used in order to ensure that the myocardium is fully in the reperfusion phase. It is also feasible that the Threshold_{REPERFUSION} may be se higher than Threshold_{PRE} to engage the post-conditioning sooner. The duration for the pacing for the post-conditioning can be variable. An exemplary duration is two minutes.

### Pacing Location:

Pacing location, for both pre-conditioning and post-conditioning periods (as well as during the ischemia itself) can be optimized by using a multipolar LV lead (e.g., Quartet™ LV lead) to pace myocardium away from the location of ischemia and infarction. Pacing-induced early-activated regions shorten considerably during early systole, thereby stretching late-activated regions with ischemia and infarction, which can induce intermittently augmented mechanical load in late-activated regions resulting in protection against the detrimental effects of ischemia and reperfusion. The pacing location can also be determined in a close-loop fashion, as already explained. For example, the pacing location that leads to the largest degree of mechanical or electrical dyssynchrony determined by the multitude of sensors mentioned above can be chosen to deliver the dyssynchrony-inducing pacing. In addition, multisite pacing in the LV can be enabled (or disabled) based on the information from the multitude of sensors mentioned above (including the AnalyST™ system).

As such, these systems and techniques generally provide for:
a) Protective pacing before and after MI or ischemia in cardiac implantable devices;
b) Determining the timing of delivery for the protective pacing using ST deviation information;
c) Determining the location of the LV pacing that can induce a cardioprotective mechanical dyssynchrony at pre- and post-conditioning periods;
d) Determining the pacing parameter that leads to the highest degree of mechanical and electrical dyssynchrony; and
e) Determining the parameters for protective pacing through a feedback system in which sensors such as cardiogenic impedance, heart sound, or PPG are used to assess the degree of dyssynchrony.

Although primarily described with respect to examples having a CRT with pacing capability (i.e. a CRT-P), other implantable medical devices may be equipped to exploit the techniques described herein such as CRT-D devices, as well as standalone pacemakers or ICDs. When exploited in a CRT, the device can exploit a wide variety of techniques to improve CRT capability. See, for example, the techniques discussed in: U.S. Published Patent Application 2010/0268059 of Ryu et al., entitled “Therapy Optimization via Multi-Dimensional Mapping” and U.S. Patent Application 2010/0152801 of Koh et al., entitled “Cardiac Resynchronization Therapy Optimization using Vector Measurements Obtained from Realtime Electrode Position Tracking.” See, also, U.S. Patent Application No. 2008/0306567 of Park et al., entitled “System and Method for Improving CRT Response and Identifying Potential Non-Responders to CRT Therapy” and U.S. Patent Application No. 2007/0179390 of Schecter, entitled “Global Cardiac Performance.”

For the sake of completeness, an exemplary pacer/CRT will now be described, which includes components for performing the functions and steps already described. Although described primarily with respect to an implementation having a quad-pole LV lead, aspects of the invention are also generally applicable to systems having other multi-pole LV leads and to systems having multi-pole RV leads or RA leads.

### Exemplary Pacer/CRT

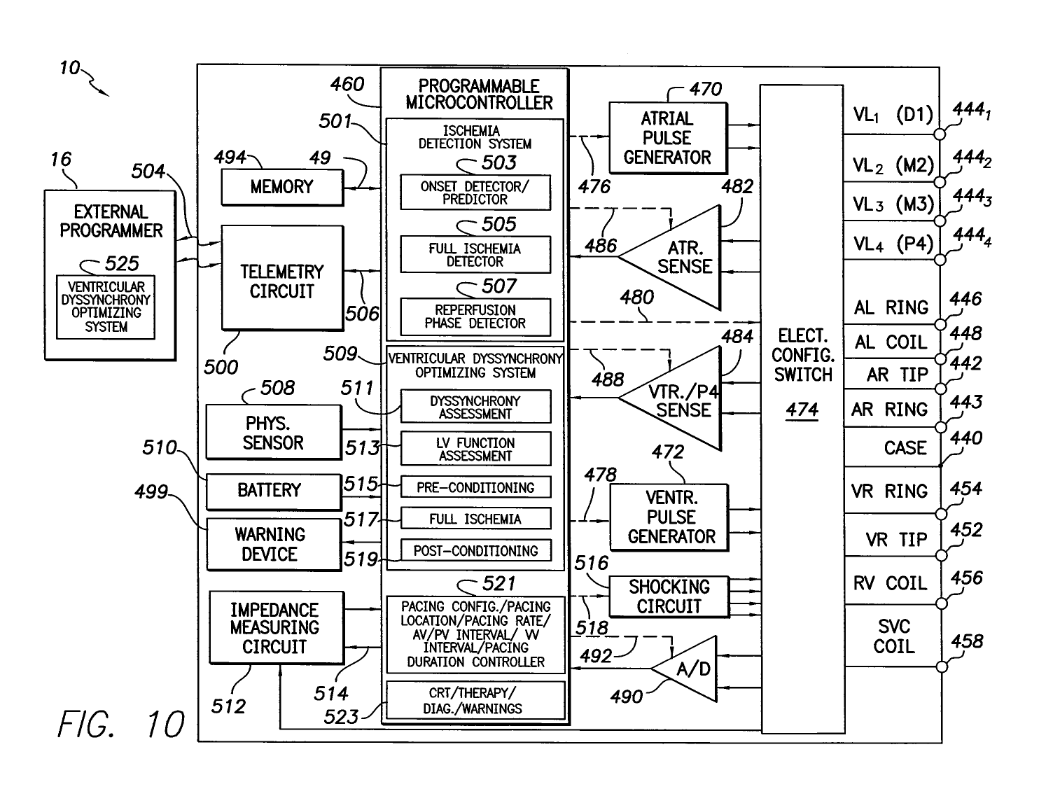

With reference to FIGS. 9 and 10, a description of an exemplary pacer/CRT will now be provided. FIG. 9 provides a simplified block diagram of the pacer/CRT, which is a dual-chamber stimulation device capable of treating both fast and slow arrhythmias with stimulation therapy, including cardioversion, defibrillation, and pacing stimulation, and also capable of controlling protective pacing, as discussed above. To provide atrial chamber pacing stimulation and sensing, pacer/CRT 10 is shown in electrical communication with a heart 412 by way of a left atrial lead 420 having an atrial tip electrode 422 and an atrial ring electrode 423 implanted in the atrial appendage. Pacer/CRT 10 is also in electrical communication with the heart by way of a right ventricular lead 430 having, in this embodiment, a ventricular tip electrode 432, a right ventricular ring electrode 434, a right ventricular (RV) coil electrode 436, and a superior vena cava (SVC) coil electrode 438. Typically, the right ventricular lead 430 is transvenously inserted into the heart so as to place the RV coil electrode 436 in the right ventricular apex, and the SVC coil electrode 438 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/CRT 10 is coupled to a multi-pole LV lead 424 designed for placement in the “CS region” via the CS os for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, the exemplary LV lead 424 is designed to receive atrial and ventricular cardiac signals and to deliver left ventricular pacing therapy using a set of four left ventricular electrodes 426₁ (D1), 426₂ (M2), 426₃ (M3), and 426₄ (P4), left atrial pacing therapy using at least a left atrial ring electrode 427, and shocking therapy using at least a left atrial coil electrode 428. The 426₁ LV electrode may also be referred to as a “tip” or “distal” LV electrode. The 426₄ LV electrode may also be referred to as a “proximal” LV electrode. In other examples, more or fewer LV electrodes are provided. Although only three leads are shown in FIG. 9, it should also be understood that additional leads (with one or more pacing, sensing and/or shocking electrodes) might be used and/or additional electrodes might be provided on the leads already shown, such as additional electrodes on the RV lead. On present commercially-available hardware, there is often no separate electrode 427. That is, the P4 electrode 426₄ and the “left atrial ring electrode” 427 are one and the same. Hence, it should be understood that the “left atrial ring electrode” could instead be used as the P4 electrode (especially if implanted on or near the AV groove.) Both electrodes are shown for the sake of completeness and generality.

A simplified block diagram of internal components of pacer/CRT 10 is shown in FIG. 10. While a particular pacer/CRT is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. The housing 440 for pacer/CRT 10, shown schematically in FIG. 10, is often referred to as the “can”, “case” or “case electrode” and may be programmably selected to act as the return electrode for all “unipolar” modes. The housing 440 may further be used as a return electrode alone or in combination with one or more of the coil electrodes, 428, 436 and 438, for shocking purposes. The housing 440 further includes a connector (not shown) having a plurality of terminals, 442, 443, 444₁-444₄, 446, 448, 452, 454, 456 and 458 (shown schematically and, for convenience, the names of the electrodes to which they are connected are shown next to the terminals). As such, to achieve right atrial sensing and pacing, the connector includes at least a right atrial tip terminal (A_{R} TIP) 442 adapted for connection to the atrial tip electrode 422 and a right atrial ring (A_{R} RING) electrode 443 adapted for connection to right atrial ring electrode 423. To achieve left chamber sensing, pacing and shocking, the connector includes a left ventricular tip terminal (VL₁ (D1)) 444₁ and additional LV electrode terminals 444₂-444₄ for the other LV electrodes of the LV lead.

The connector also includes a left atrial ring terminal (A_{L} RING) 446 and a left atrial shocking terminal (A_{L} COIL) 448, which are adapted for connection to the left atrial ring electrode 427 and the left atrial coil electrode 428, respectively. To support right chamber sensing, pacing and shocking, the connector further includes a right ventricular tip terminal (V_{R} TIP) 452, a right ventricular ring terminal (V_{R} RING) 454, a right ventricular shocking terminal (RV COIL) 456, and an SVC shocking terminal (SVC COIL) 458, which are adapted for connection to the right ventricular tip electrode 432, right ventricular ring electrode 434, the V_{R} coil electrode 436, and the SVC coil electrode 438, respectively.

At the core of pacer/CRT 10 is a programmable microcontroller 460, which controls the various modes of stimulation therapy. As is well known in the art, the microcontroller 460 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 460 includes the ability to process or monitor input signals (data) as controlled by a program code stored in a designated block of memory. The details of the design and operation of the microcontroller 460 are not critical to the invention. Rather, any suitable microcontroller 460 may be used that carries out the functions described herein. The use of microprocessor-based control circuits for performing timing and data analysis functions are well known in the art.

As shown in FIG. 10, an atrial pulse generator 470 and a ventricular pulse generator 472 generate pacing stimulation pulses for delivery by the right atrial lead 420, the right ventricular lead 430, and/or the LV lead 424 via an electrode configuration switch 474. It is understood that in order to provide stimulation therapy in each of the four chambers of the heart, the atrial and ventricular pulse generators, 470 and 472, may include dedicated, independent pulse generators, multiplexed pulse generators or shared pulse generators. The pulse generators, 470 and 472, are controlled by the microcontroller 460 via appropriate control signals, 476 and 478, respectively, to trigger or inhibit the stimulation pulses.

The microcontroller 460 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 474 includes a plurality of switches for connecting the desired electrodes to the appropriate I/O circuits, thereby providing complete electrode programmability. Accordingly, the switch 474, in response to a control signal 480 from the microcontroller 460, determines the polarity of the stimulation pulses (e.g., unipolar, bipolar, combipolar, etc.) by selectively closing the appropriate combination of switches (not shown) as is known in the art. The switch also switches among the various LV electrodes.

Atrial sensing circuits 482 and ventricular sensing circuits 484 may also be selectively coupled to the right atrial lead 420, LV lead 424, and the right ventricular lead 430, through the switch 474 for detecting the presence of cardiac activity in each of the four chambers of the heart. The ventricular sense circuit preferably accommodates at least one P4 sensing channel. Accordingly, the atrial (ATR. SENSE) and ventricular (VTR. SENSE) sensing circuits, 482 and 484, may include dedicated sense amplifiers, multiplexed amplifiers or shared amplifiers. The switch 474 determines the “sensing polarity” of the cardiac signal by selectively closing the appropriate switches, as is also known in the art. In this way, the clinician may program the sensing polarity independent of the stimulation polarity. Each sensing circuit, 482 and 484, preferably employs one or more low power, precision amplifiers with programmable gain and/or automatic gain control, bandpass filtering, and a threshold detection circuit, as known in the art, to selectively sense the cardiac signal of interest. The automatic gain control enables pacer/CRT 10 to deal effectively with the difficult problem of sensing the low amplitude signal characteristics of atrial or ventricular fibrillation. The outputs of the atrial and ventricular sensing circuits, 482 and 484, are connected to the microcontroller 460 which, in turn, are able to trigger or inhibit the atrial and ventricular pulse generators, 470 and 472, respectively, in a demand fashion in response to the absence or presence of cardiac activity in the appropriate chambers of the heart.

For arrhythmia detection, pacer/CRT 10 utilizes the atrial and ventricular sensing circuits, 482 and 484, to sense cardiac signals to determine whether a rhythm is physiologic or pathologic. As used in this section “sensing” is reserved for the noting of an electrical signal, and “detection” is the processing of these sensed signals and noting the presence of an arrhythmia. The timing intervals between sensed events (e.g., AS, VS, and depolarization signals associated with fibrillation which are sometimes referred to as “F-waves” or “Fib-waves”) are then classified by the microcontroller 460 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (ND) data acquisition system 490. The data acquisition system 490 is configured to acquire intracardiac electrogram signals, convert the raw analog data into a digital signal, and store the digital signals for later processing and/or telemetric transmission to an external device 16. The data acquisition system 490 is coupled to the right atrial lead 420, the LV lead 424, and the right ventricular lead 430 through the switch 474 to sample cardiac signals across any pair of desired electrodes. The microcontroller 460 is further coupled to a memory 494 by a suitable data/address bus 496, wherein the programmable operating parameters used by the microcontroller 460 are stored and modified, as required, in order to customize the operation of pacer/CRT 10 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/CRT 10 may be non-invasively programmed into the memory 494 through a telemetry circuit 500 in telemetric communication with the external device 16, such as a programmer, transtelephonic transceiver or a diagnostic system analyzer. The telemetry circuit 500 is activated by the microcontroller by a control signal 506. The telemetry circuit 500 advantageously allows intracardiac electrograms and status information relating to the operation of pacer/CRT 5 (as contained in the microcontroller 460 or memory 494) to be sent to the external device 502 through an established communication link 504. Pacer/CRT 10 further includes an accelerometer or other physiologic sensor 508, commonly referred to as a “rate-responsive” sensor because it is typically used to adjust pacing stimulation rate according to the exercise state of the patient. However, the physiological sensor 508 may further be used to detect changes in cardiac output, changes in the physiological condition of the heart, or diurnal changes in activity (e.g., detecting sleep and wake states) and to detect arousal from sleep. Accordingly, the microcontroller 460 responds by adjusting the various pacing parameters (such as rate, AV delay, VV delay, etc.) at which the atrial and ventricular pulse generators, 470 and 472, generate stimulation pulses. While shown as being included within pacer/CRT 10, it is to be understood that the physiologic sensor 508 may also be external to pacer/CRT 10, yet still be implanted within or carried by the patient. A common type of rate responsive sensor is an activity sensor incorporating an accelerometer or a piezoelectric crystal, which is mounted within the housing 440 of pacer/CRT 10. Other types of physiologic sensors are also known, for example, sensors that sense the oxygen content of blood, respiration rate and/or minute ventilation, pH of blood, ventricular gradient, contractility, mechanical dyssynchrony, electrical dyssynchrony, PPG, LAP, PAP, heart sounds, etc.

The pacer/CRT additionally includes a battery 510, which provides operating power to all of the circuits shown in FIG. 10. The battery 510 may vary depending on the capabilities of pacer/CRT 10. If the system only provides low voltage therapy, a lithium iodine or lithium copper fluoride cell typically may be utilized. For pacer/CRT 10, which employs shocking therapy, the battery 510 should be capable of operating at low current drains for long periods, and then be capable of providing high-current pulses (for capacitor charging) when the patient requires a shock pulse. The battery 510 should also have a predictable discharge characteristic so that elective replacement time can be detected. Accordingly, appropriate batteries are employed.

As further shown in FIG. 10, pacer/CRT 10 is shown as having an impedance measuring circuit 512, which is enabled by the microcontroller 460 via a control signal 514. Uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, and detecting cardiogenic impedance, etc. The impedance measuring circuit 512 is advantageously coupled to the switch 474 so that any desired electrode may be used.

In the case where pacer/CRT 10 is intended to operate as an ICD device, it detects the occurrence of an arrhythmia, and automatically applies an appropriate electrical shock therapy to the heart aimed at terminating the detected arrhythmia. To this end, the microcontroller 460 further controls a shocking circuit 516 by way of a control signal 518. The shocking circuit 516 generates shocking pulses of low (up to 0.5 joules), moderate (0.5-10 joules) or high energy (11 to 40 joules or more), as controlled by the microcontroller 460. Such shocking pulses are applied to the heart of the patient through at least two shocking electrodes, and as shown in this embodiment, selected from the left atrial coil electrode 428, the RV coil electrode 436, and/or the SVC coil electrode 438. The housing 440 may act as an active electrode in combination with the RV electrode 436, or as part of a split electrical vector using the SVC coil electrode 438 or the left atrial coil electrode 428 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 10-40 joules or more), delivered asynchronously (since R-waves may be too disorganized), and pertaining exclusively to the treatment of fibrillation. Accordingly, the microcontroller 460 is capable of controlling synchronous or asynchronous delivery of shocking pulses.

An internal warning device 499 may be provided for generating perceptible warning signals to the patient via vibration, voltage or other methods.

Insofar as protective pacing is concerned, the microcontroller includes an ischemia detection system 501 operative to sense cardioelectric signals within the heart using the leads and analyze the cardioelectric signals to detect an indication of an ischemic event within the heart. In this example, the ischemia detection system includes: an ischemia onset predictor/detector 503 operative to detect early onset of ischemia and thereby predict the subsequent full ischemia; a full ischemia detector 505 operative to detect full ischemia; and a reperfusion phase detector 507 operative to detect the reperfusion phase.

The microcontroller also includes a ventricular dyssynchrony optimizing system 509 operative to control the delivery of pacing stimulus in accordance with adjustable pacing parameters to induce ventricular dyssynchrony within the heart and further operative to adjust the pacing parameters within a range of permissible values while pacing stimulus is delivered to achieve a preferred degree of ventricular dyssynchrony within the patient. In this example, the dyssynchrony optimizing system includes: a dyssynchrony assessment system 511 operative to assess mechanical and/or electrical dyssynchrony; an LV function assessment system 513 operative to assess LV pumping functionality; a pre-conditioning pacing controller 515 operative to control pre-condition pacing; a full ischemia pacing controller 517 operative to control delivery of protective pacing during a full ischemia; and a post-condition pacing controller 519 operative to control post-conditioning pacing. To adjust pacing parameters during protective pacing to maximize dyssynchrony (or for other reasons), the microcontroller also includes a pacing parameter controller 512 operative to control one or more of pacing configuration, pacing location, pacing rate, AV/PV interval, VV interval, interpulse intervals, and pacing duration, as already described above.

Therapy, diagnostics and warnings are controlled by system 523, which also controls delivery of CRT, where appropriate.

Depending upon the implementation, the various components of the microcontroller may be implemented as separate software modules or the modules may be combined to permit a single module to perform multiple functions. In addition, although shown as being components of the microcontroller, some or a10 of these components may be implemented separately from the microcontroller, using application specific integrated circuits (ASICs) or the like. At least some of the techniques described herein can be performed by (or under the control of) an external device, such as programmer 16, subject to clinician instructions. Accordingly, the external device is shown as including a ventricular dyssynchrony optimizing system 525.

In general, while the invention has been described with reference to particular embodiments, modifications can be made thereto without departing from the scope of the invention. Note also that the term “including” as used herein is intended to be inclusive, i.e. “including but not limited to.”

What is claimed is:

1. A method for use with an implantable medical device having leads for sensing and pacing within the heart, the method comprising:
sensing cardioelectric signals within the heart using the leads and analyzing the cardioelectric signals to detect an indication of an ischemic event within the heart;
delivering pacing stimulus in accordance with adjustable pacing parameters to induce ventricular dyssynchrony within the heart in response to detection of the indication of the ischemic event; and
adjusting the pacing parameters within a range of permissible values while pacing stimulus is delivered to achieve a preferred degree of ventricular dyssynchrony within the patient.

2. The method of claim 1 wherein analyzing the cardioelectric signals to detect an indication of an ischemic event includes:
detecting full onset of an ischemic event.

3. The method of claim 1 wherein analyzing the cardioelectric signals to detect an indication of an ischemic event includes:
detecting early onset of an ischemic event.

4. The method of claim 3 wherein analyzing the cardioelectric signals to detect early onset of the ischemic event includes:
identifying ST segments within the cardioelectric signals and assessing ST segment elevation; and
detecting a deviation in the ST segment elevation indicative of early onset of the ischemic event.

5. The method of claim 4 wherein detecting a deviation in the ST segment elevation indicative of early onset of the ischemic event includes:
inputting first and second ST segment change thresholds, the first ST threshold (Threshold_{PRE}) indicative of early onset of ischemia, the second ST threshold (Threshold_{FULL}) indicative of full onset of ischemia; and
detecting the early onset of the ischemic event when the deviation in ST segment elevation exceeds the first threshold.

6. The method of claim 3 further including detecting full onset of ischemia and wherein the delivery of pacing stimulus to induce ventricular dyssynchrony is suspended upon detection of full ischemia.

7. The method of claim 6 further including detecting the end of full ischemia and wherein the delivery of pacing stimulus to induce ventricular dyssynchrony resumes following the end of full ischemia during a reperfusion interval.

8. The method of claim 7 further including detecting the reperfusion interval using a reperfusion threshold (Threshold_{REPERFUSION}) set lower than a threshold (Threshold_{FULL}) indicative of full onset of ischemia.

9. The method of claim 3 further including detecting full onset of ischemia and wherein the delivery of pacing stimulus to induce ventricular dyssynchrony continues during full ischemia.

10. The method of claim 1 wherein the device monitors cardiac conditions and, if cardiac conditions deteriorate beyond an acceptable level, delivery of pacing stimulus to induce ventricular dyssynchrony is suspended.

11. The method of claim 10 wherein monitoring cardiac conditions includes monitoring left ventricular (LV) pumping functionality and wherein, if LV pumping functionality deteriorates below an acceptable level, delivery of pacing stimulus to induce ventricular dyssynchrony is suspended.

12. The method of claim 11 wherein LV contractility is monitored to assess LV pumping functionality.

13. The method of claim 12 wherein LV contractility is monitored using one or more of: heart sounds, cardiogenic impedance, photoplethysmography (PPG) signals, pulmonary artery pressure (PAP) signals and left atrial pressure (LAP) signals.

14. The method of claim 1 wherein the adjustable pacing parameters include parameters specify one or more of: a pacing configuration; a pacing location; a pacing rate; an atrioventricular (AV/PV) interval; an interventricular (VV) interval; interpulse intervals for multi-site LV pacing; a duration of sequences of the pacing stimulus; pacing vector combinations; and number of pacing electrodes.

15. The method of claim 14 wherein adjusting the parameters while pacing stimulus is delivered includes:
assessing the degree of ventricular dyssynchrony within the patient while stimulus is ongoing; and
adjusting the parameters within a feedback loop to determine a combination of parameters sufficient to maximize the degree of ventricular dyssynchrony.

16. The method of claim 15 wherein assessing the degree of ventricular dyssynchrony includes assessing one or both of mechanical dyssynchrony and electrical dyssynchrony.

17. The method of claim 16 wherein assessing the degree of ventricular dyssynchrony includes assessing one or more of: a change in activation interval (AI); an increase in preload; and a widening of a QRS interval within the cardioelectric signals.

18. The method of claim 17 wherein the assessment is made based on one or more of cardiogenic impedance signals, heart sound signals, photoplethysmography (PPG) signals, PAP signals and LAP signals detected using implanted sensors.

19. The method of claim 14 wherein the parameters for controlling pacing location are adjusted to pace the myocardium at a location away from the location of an ischemia.

20. The method of claim 1 wherein the pacing stimulus is delivered at a rate sufficient to substantially eliminate intrinsic myocardial activation during pacing.

21. The method of claim 20 wherein the pacing stimulus is delivered using atrioventricular (AV/PV) delays sufficiently short to permit dyssynchronous ventricular activation.

22. The method of claim 1 wherein a10 of the steps are performed by the implantable medical device.

23. The method of claim 1 wherein at least some of the steps are performed by an external device based on signals received from the implantable medical device.

24. A system for use with an implantable medical device having leads for sensing and pacing within the heart, the system comprising:
an ischemia detection system operative to sense cardioelectric signals within the heart using the leads and analyze the cardioelectric signals to detect an indication of an ischemic event within the heart;
a pacing stimulus controller operative to deliver pacing stimulus in accordance with adjustable pacing parameters; and
a ventricular dyssynchrony maximizing system operative to control the delivery of pacing stimulus in accordance with adjustable pacing parameters to induce ventricular dyssynchrony within the heart and further operative to adjust the pacing parameters within a range of permissible values while pacing stimulus is delivered to achieve a preferred degree of ventricular dyssynchrony within the patient.

25. A system for use with an implantable medical device having leads for sensing and pacing within the heart, the system comprising:
means for sensing cardioelectric signals within the heart using the leads;
means for analyzing the cardioelectric signals to detect an indication of an ischemic event within the heart;
means for delivering pacing stimulus in accordance with adjustable pacing parameters to induce ventricular dyssynchrony within the heart in response to detection of the indication of the ischemic event; and
means for adjusting the pacing parameters within a range of permissible values while pacing stimulus is delivered to achieve a preferred degree of ventricular dyssynchrony within the patient.
